(19) United States
(12) Patent Application Publication
(10) Pub. No.: US 2025/0336217 A1
(43) Pub. Date: Oct. 30, 2025

(54) DETERMINING DEVICE, STORAGE MEDIUM STORING COMPUTER PROGRAM FOR DETERMINATION, AND DETERMINING METHOD

(71) Applicants: TOYOTA JIDOSHA KABUSHIKI KAISHA, Toyota-shi Aichi-ken (JP); DENSO CORPORATION, Kariya-city Aichi-pref (JP)

(72) Inventors: Kenichiroh Hara, Yokohama-shi Kanagawa-ken (JP); Yutaro Tsugawa, Edogawa-ku Tokyo-to (JP); Hiromi Nemoto, Shinagawa-ku Tokyo-to (JP); Jiro Fujino, Nerima-ku Tokyo-to (JP); Mamoru Sakura, Koto-ku Tokyo-to (JP)

(73) Assignees: TOYOTA JIDOSHA KABUSHIKI KAISHA, Toyota-shi Aichi-ken (JP); DENSO CORPORATION, Kariya-city Aichi-pref (JP)

(21) Appl. No.: 19/184,492

(22) Filed: Apr. 21, 2025

(30) Foreign Application Priority Data
Apr. 24, 2024 (JP) 2024-070928

(51) Int. Cl. G06V 20/59; A61B 5/18; G06V 40/16; G06V 40/18; G06V 40/20

(52) U.S. Cl. CPC G06V 20/597; A61B 5/18; G06V 40/16; G06V 40/18; G06V 40/20

(57) ABSTRACT

A determining device has a processor configured to determine that a driver has not dozed off when it has been determined that a degree of eye opening of the driver is at or below a reference degree of eye opening and a direction of a driver's face indicates movement in a right direction exceeding a first reference and, in continuation with the movement, the direction of the driver's face indicates movement in a left direction exceeding a second reference, and determine that the driver has dozed off when the degree of eye opening is at or below the reference degree of eye opening and it has not been determined that the direction of the driver's face indicates movement in the right direction exceeding the first reference and, in continuation with the movement, the direction of the driver's face indicates movement in the left direction exceeding the second reference.

### CROSS-REFERENCE TO RELATED APPLICATIONS

This application claims priority to Japanese Patent Application No. 2024-070928 filed Apr. 24, 2024, the entire contents of which are herein incorporated by reference.

### FIELD

The present disclosure relates to a determining device, a storage medium storing a computer program for determination, and a determining method.

### BACKGROUND

A monitoring device mounted in a vehicle has conventionally been used to monitor a driver's state. For example, a monitoring device may monitor whether a driver is dozing off.

The monitoring device determines whether the driver is dozing off by detecting the degree of eye opening based on an image of the driver's face. When the monitoring device has detected that a state in which the degree of eye opening is low (a state of closed eyes) has continued for a predetermined time period, it is determined that the driver has dozed off, and the driver is notified with a warning.

Japanese Unexamined Patent Publication No. 2011-048531, for example, proposes a technique in which dozing off of a driver is determined based on eyelid movement, and on changes in the face angle immediately after eyelid movement. In Japanese Unexamined Patent Publication No. 2011-048531, it is determined that the eyes are not sleepy if the face angle has moved at least a predetermined amount to the left or right immediately after the eyelids have closed.

### SUMMARY

However, since a driver may fall asleep with the face directed in one particular direction, in some cases it may not be possible to detect dozing off of the driver by the technique proposed in Japanese Unexamined Patent Publication No. 2011-048531.

Moreover in some situations the degree of eye opening may fall below the threshold while the driver is smiling. It is not always possible to accurately detect that the driver is smiling simply by detecting that the face angle has moved at least a predetermined amount to the left or right immediately after the eyelids have closed.

It is therefore an object of the present disclosure to provide a determining device that can determine whether a driver has dozed off without erroneously detecting a smiling face.
(1) According to one embodiment, the present disclosure provides a determining device. The determining device has a processor configured to determine whether a degree of eye opening of a driver is at or below a reference degree of eye opening, determine whether a direction of a driver's face indicates movement in a right direction exceeding a first reference and, in continuation with the movement in the right direction, the direction of the driver's face indicates movement in a left direction exceeding a second reference, based on information representing movement of the driver's face, and determine that the driver has not dozed off when it has been determined that the degree of eye opening of the driver is at or below the reference degree of eye opening and it has been determined that the direction of the driver's face indicates movement in the right direction exceeding the first reference and, in continuation with the movement in the right direction, the direction of the driver's face indicates movement in the left direction exceeding the second reference, and determine that the driver has dozed off when it has been determined that the degree of eye opening of the driver is at or below the reference degree of eye opening and it has not been determined that the direction of the driver's face indicates movement in the right direction exceeding the first reference and, in continuation with the movement in the right direction, the direction of the driver's face indicates movement in the left direction exceeding the second reference.
(2) In the determining device of the embodiment (1) above, the information representing movement of the driver's face is represented by an angle of the direction of the driver's face in a horizontal direction, angular velocity of the direction of the driver's face in the horizontal direction, or angular acceleration of the direction of the driver's face in the horizontal direction.
(3) In the determining device of the embodiment (1) or (2), the processor is further configured to determine that the driver has not dozed off when it has been determined that the direction of the driver's face indicates movement in the right direction exceeding the first reference and, in continuation with the movement in the right direction, the direction of the driver's face indicates movement in the left direction exceeding the second reference, within a reference time from the time at which it has determined that the driver has not dozed off.
(4) In the determining device of any one of the embodiments (1) to (3), the processor is further configured to determine whether the direction of the driver's face indicates movement in an upward direction exceeding a third reference or the direction of the driver's face indicates movement in a downward direction exceeding a fourth reference, based on movement of the driver's face, and determine that the driver has dozed off when it has been determined that the degree of eye opening of the driver is at or below the reference degree of eye opening and it has been determined that the direction of the driver's face indicates movement in the upward direction exceeding the third reference or the direction of the driver's face indicates movement in the downward direction exceeding the fourth reference, even when it has been determined that the direction of the driver's face indicates movement in the right direction exceeding the first reference and, in continuation with the movement in the right direction, the direction of the driver's face indicates movement in the left direction exceeding the second reference.
(5) According to another embodiment, a storage medium storing a computer program for determination is provided. The computer program for determination includes determining whether a degree of eye opening of a driver is at or below a reference degree of eye opening, determining whether a direction of a driver's face indicates movement in a right direction exceeding a first reference and, in continuation with the movement in the right direction, the direction of the driver's face indicates movement in a left direction exceeding a second reference, based on information representing movement of the driver's face, and determining that the driver has not dozed off when it has been determined that the degree of eye opening of the driver is at or below the reference degree of eye opening and it has been determined that the direction of the driver's face indicates movement in the right direction exceeding the first reference and, in continuation with the movement in the right direction, the direction of the driver's face indicates movement in the left direction exceeding the second reference, and determining that the driver has dozed off when it has been determined that the degree of eye opening of the driver is at or below the reference degree of eye opening and it has not been determined that the direction of the driver's face indicates movement in the right direction exceeding the first reference and, in continuation with the movement in the right direction, the direction of the driver's face indicates movement in the left direction exceeding the second reference.
(6) According to yet another embodiment of the present disclosure, a determining method is provided. The determining method includes determining whether a degree of eye opening of a driver is at or below a reference degree of eye opening, determining whether a direction of a driver's face indicates movement in a right direction exceeding a first reference and, in continuation with the movement in the right direction, the direction of the driver's face indicates movement in a left direction exceeding a second reference, based on information representing movement of the driver's face, and determining that the driver has not dozed off when it has been determined that the degree of eye opening of the driver is at or below the reference degree of eye opening and it has been determined that the direction of the driver's face indicates movement in the right direction exceeding the first reference and, in continuation with the movement in the right direction, the direction of the driver's face indicates movement in the left direction exceeding the second reference, and determining that the driver has dozed off when it has been determined that the degree of eye opening of the driver is at or below the reference degree of eye opening and it has not been determined that the direction of the driver's face indicates movement in the right direction exceeding the first reference and, in continuation with the movement in the right direction, the direction of the driver's face indicates movement in the left direction exceeding the second reference.

The determining device of the present disclosure can determine that a driver has dozed off without erroneously detecting a smiling face.

The object and advantages of the present disclosure will be realized and attained by the elements and combinations particularly specified in the claims. It is to be understood that both the foregoing general description and the following detailed description are exemplary and explanatory and are not restrictive of the present disclosure, as claimed.

### BRIEF DESCRIPTION OF DRAWINGS

FIG. 1 is a diagram illustrating operation of a monitoring device of the first embodiment in overview.

FIG. 2 is a general schematic drawing of a vehicle in which the monitoring device of the first embodiment is mounted.

FIG. 3 is an example of an operation flow chart for monitoring processing by the monitoring device of the first embodiment.

FIG. 4 is a diagram showing change in facial direction representing movement of the driver's face.

FIG. 5 is a diagram showing change in angular velocity representing movement of the driver's face.

FIG. 6 is an example of an operation flow chart for monitoring processing by the monitoring device of the second embodiment.

FIG. 7 is another example of an operation flow chart for monitoring processing by the monitoring device of the third embodiment.

### DESCRIPTION OF EMBODIMENTS

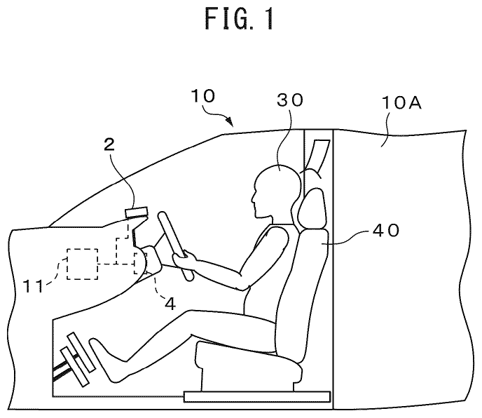

FIG. 1 is a diagram illustrating operation of a monitoring device 11 of the first embodiment in overview. The vehicle 10 has the monitoring device 11, as shown in FIG. 1. The driver 30 sits in a driving seat 40 in the cabin 10A of the vehicle 10. A monitoring camera 2 is disposed on the dashboard.

The monitoring device 11 monitors the driver 30, based on monitor images acquired by the monitoring camera 2. The monitoring device 11 is an example of a determining device. The vehicle 10 may also be a self-driving vehicle.

The monitoring camera 2 is able to acquire images representing the face of the driver 30. The monitoring device 11 detects the degree of eye opening of the driver 30 based on monitor images acquired by the monitoring camera 2. The monitoring device 11 also detects information representing movement of the face of the driver 30, based on the monitor images.

The monitoring device 11 determines whether or not the driver 30 is dozing off based on information representing the degree of eye opening of the driver 30 and movement of the face of the driver 30. When it has been determined that the driver 30 has dozed off, the monitoring device 11 gives the driver 30 a notification warning via a user interface (UI) 4.

The monitoring device 11 determines whether or not the eyes of the driver 30 are closed based on the degree of eye opening of the driver 30. When the driver 30 is smiling, however, it could be detected that a lower degree of eye opening has continued for a predetermined period of time. In some embodiments, in order to prevent erroneous determination that the driver has dozed off it is desirable to be able to accurately determine whether the driver is smiling or has dozed off.

Based on survey results, the present inventors have found that humans continuously change the left and right facial direction when smiling. When a human is sleeping, the face may be oriented in one direction for resting of the head, but the facial direction does not change in a continuous manner to the left and right while sleeping. Continuously changing the face direction to the left and right is a feature associated with human smiling.

Therefore when it has been determined that the eyes of the driver 30 are closed, the monitoring device 11 determines whether or not the direction of the face is continuously changing to the left and right, based on information representing movement of the face of the driver 30.

When it has been determined that the degree of eye opening of the driver 30 is at or below a reference degree of eye opening, the monitoring device 11 examines movement of the face of the driver 30. The monitoring device 11 determines that the driver 30 has not dozed off when it has been determined that the direction of the face of the driver 30 indicates movement in the right direction exceeding the first reference and, in continuation with the movement in the right direction, the direction of the face of the driver 30 indicates movement in the left direction exceeding the second reference.

Even if it has been determined that the eyes of the driver 30 are closed, the driver 30 is smiling if the direction of the face is continuously changing to the left and right. Determining whether or not the direction of the face is continuously changing to the left and right prevents smiling by the driver 30 from being erroneously judged as having dozed off.

When it has been determined that the degree of eye opening of the driver 30 is at or below the reference degree of eye opening, and it has not been determined that the direction of the face of the driver 30 indicates movement in the right direction exceeding the first reference and, in continuation with the movement in the right direction, the direction of the face of the driver 30 indicates movement in the left direction exceeding the second reference, then the monitoring device 11 determines that the driver 30 has dozed off.

In other words, when it has been determined that the eyes of the driver 30 are closed and the facial direction has not changed continuously to the left and right, the driver 30 is estimated to have dozed off.

When it has been determined that the driver 30 has dozed off, the monitoring device 11 gives the driver 30 a notification warning via the UI 4, to increase the degree of participation in driving.

As explained above, the monitoring device 11 of the embodiment can determine that the driver 30 has dozed off without erroneously detecting a smiling face.

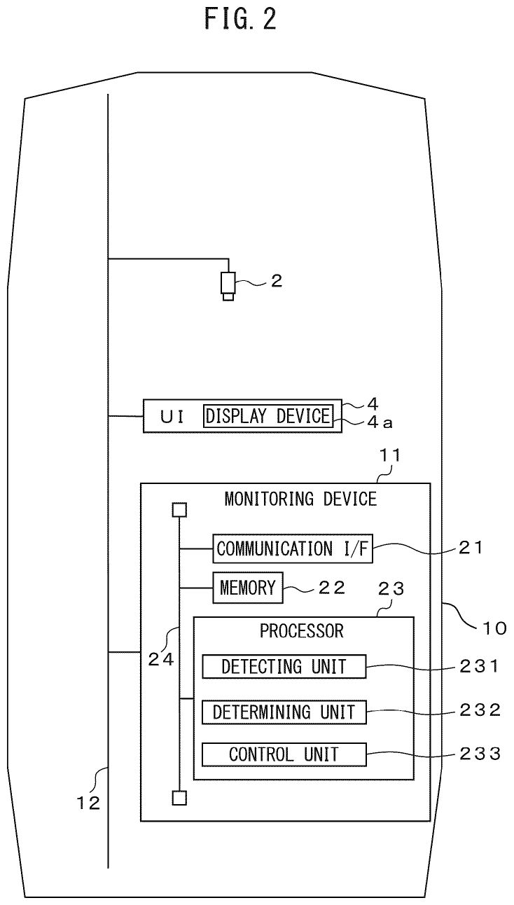

FIG. 2 is a general schematic drawing of a vehicle 10 in which the monitoring device 11 of the embodiment is mounted. The vehicle 10 has a monitoring camera 2, a user interface (UI) 4 and a monitoring device 11, etc.

The monitoring camera 2, UI 4 and monitoring device 11 are connected in a communicable manner via an in-vehicle network 12 conforming to the Controller Area Network standard.

The monitoring camera 2 is disposed inside the cabin 10A in a manner allowing the monitoring camera 2 to acquire monitor images including the face of the driver 30 driving the vehicle 10. The monitoring camera 2 is an example of an image acquisition unit. For example, the monitoring camera 2 is disposed so as to allow the monitoring camera 2 to acquire images near the driving seat 40. The monitoring camera 2 is disposed on the dashboard, for example, as shown in FIG. 1.

For example, the monitoring camera 2 acquires monitor images at a monitor image acquisition time having a predetermined cycle. Each time a monitor image is acquired, the monitoring camera 2 outputs the monitor image and the image acquisition time to the monitoring device 11 via the in-vehicle network 12. The predetermined cycle may be 0.1 to 0.5 seconds, for example.

The monitoring camera 2 has a 2D detector composed of an array of photoelectric conversion elements with infrared sensitivity, such as a CCD or C-MOS, and an imaging optical system that forms an image of the photographed region on the 2D detector. In some embodiments, the monitoring camera 2 has a lighting device in addition to the 2D detector. The lighting device, for example, consists of two near-infrared LEDs situated on either side of the imaging optical system. Illuminating the driver 30 with near-infrared light allows the face of the driver 30 to be imaged without causing discomfort for the driver 30 even during low-illuminance periods such as nighttime.

The UI 4 is an example of the notification unit. The UI 4, controlled by the monitoring device 11, notifies the driver 30 with a warning, for example. The UI 4 has a display device 4a such as a liquid crystal display or touch panel, for display of the warning. The UI 4 may also have an acoustic output device (not shown) to notify the driver 30 of the warning. The UI 4 also has a touch panel or operating button, for example, as an input device for inputting operation information from the driver 30 to the vehicle 10. The UI 4 outputs the input operation information to the monitoring device 11 via the in-vehicle network 12.

The monitoring device 11 carries out detection processing, determination processing and control processing. For this purpose, the monitoring device 11 has a communication interface (IF) 21, a memory 22 and a processor 23. The communication interface 21, memory 22 and processor 23 are connected via signal wires 24. The communication interface 21 has interface circuitry to connect the monitoring device 11 with the in-vehicle network 12.

The memory 22 is an example of a storage unit, and it has a volatile semiconductor memory and a non-volatile semiconductor memory, for example. The memory 22 stores an application computer program and various data to be used for information processing carried out by the processor 23 of each device. The memory 22 also stores monitor images input from the monitoring camera 2, in association with the image acquisition times.

All or some of the functions of the monitoring device 11 are functional modules driven by a computer program operating on the processor 23, for example. The processor 23 has a detecting unit 231, a determining unit 232 and a control unit 233. The determining unit 232 is an example of the first determining unit, second determining unit and third determining unit. Alternatively, the functional module of the processor 23 may be a specialized computing circuit in the processor 23.

The processor 23 has one or more CPUs (Central Processing Units) and their peripheral circuits. The processor 23 may also have other computing circuits such as a logical operation unit, numerical calculation unit or graphics processing unit. The driver monitoring device 11 is an electronic control unit (ECU), for example.

The detecting unit 231 calculates the degree of eye opening of the driver 30 based on multiple monitor images, at an eye opening degree detection time having a predetermined cycle. The cycle may be from 0.1 to 5 seconds, for example.

The detecting unit 231 has a classifier trained to detect eye regions by input of monitor images. The classifier inputs monitor images and detects the eye regions within the monitor images.

The classifier is a deep neural network (DNN) having multiple layers connected in series from the input end to the output end, for example. Images including the eyes are previously input into the DNN as teacher data for learning, whereby the DNN is able to function as a classifier to detect eye regions. A machine learning model such as a support vector machine or random forest may also be used as the classifier.

The detecting unit 231 also calculates the distance from the monitoring camera 2 to the face of the driver 30. For example, the detecting unit 231 refers to the relationship between distances and standard sizes of human faces represented in images, and calculates the distance between the monitoring camera 2 and the face of the driver 30 based on the magnitude of the face of the driver 30 represented in the monitor image. The region of the face of the driver 30 in the monitor image is detected using a classifier that has been trained to detect face regions.

The detecting unit 231 calculates the degree of eye opening based on the distance from the monitoring camera 2 to the face of the driver 30 and a number of pixels between the upper eyelid and lower eyelid in the monitor image. For example, the detecting unit 231 may calculate the maximum number of pixels between the upper eyelid and lower eyelid. The degree of eye opening is represented in mm units, for example. The detecting unit 231 relays the degree of eye opening for the right eye and left eye of the driver to the determining unit 232.

The detecting unit 231 detects information representing movement of the face of the driver 30, based on monitor images taken at an information detection time having a predetermined cycle. The cycle may be from 0.1 to 1 second, for example. The detecting unit 231 relays the information representing the movement of the face of the driver 30 to the determining unit 232.

The information representing movement of the face of the driver 30 is represented, for example, by the angle of the direction of the face of the driver 30 in the horizontal direction, the angular velocity of the direction of the face of the driver 30 in the horizontal direction, or the angular acceleration of the direction of the face of the driver 30 in the horizontal direction.

The direction of the face of the driver 30 is represented, for example, by the angle in the horizontal direction between the traveling direction of the vehicle 10 and the direction in which the face of the driver 30 is facing. For example, when the traveling direction of the vehicle 10 is 0°, the direction of the face of the driver 30 when facing left is represented as an angle between 0° and −180°, while the direction of the face of the driver 30 when facing right is represented as an angle between 0° and 180°.

The detecting unit 231 has a classifier that has been trained to detect facial aspects such as eye corners, inner eye corners and mouth angles from images. The detecting unit 231 inputs monitor images into the classifier to determine the locations of predetermined facial aspects in the monitor images. The detecting unit 231 also compares the locations of the predetermined facial aspects detected from the monitor image against a standard facial three-dimensional model. The angle of the face in a three-dimensional model in which the location of each facial aspect maximally matches the aspect location detected from the monitor image is detected as the angle of the face in the monitor image.

The classifier may be a deep neural network (DNN) having multiple layers connected in series from the input end to the output end, for example. Facial images including predetermined facial aspects are previously input into the DNN as teacher data for learning, whereby the DNN functions as a classifier to identify the locations of predetermined facial aspects.

The detecting unit 231 obtains a time series change for the angle of the direction of the face of the driver 30 in the horizontal direction. The detecting unit 231 may also calculate the direction of the face of the driver 30 based on the line of sight direction of the driver 30.

The detecting unit 231 may also calculate the angular velocity of the direction of the face of the driver 30 in the horizontal direction as the amount of change per unit time in the angle of the direction of the face of the driver 30 in the horizontal direction. The detecting unit 231 thus obtains a time series change for the angular velocity of the direction of the face of the driver 30 in the horizontal direction.

The detecting unit 231 may also calculate the angular acceleration of the direction of the face of the driver 30 in the horizontal direction as the amount of change per unit time in the angular velocity of the direction of the face of the driver 30 in the horizontal direction. The detecting unit 231 thus obtains a time series change for the angular acceleration of the direction of the face of the driver 30 in the horizontal direction.

The detecting unit 231 may also detect movement of the face of the driver 30 in the vertical direction. Movement of the direction of the face of the driver 30 in the vertical direction is represented, for example, by the angle in the vertical direction between the traveling direction of the vehicle 10 and the direction in which the face of the driver 30 is facing. The detecting unit 231 may also calculate the angular velocity and angular acceleration of the direction of the face of the driver 30 in the vertical direction.

For example, when the traveling direction of the vehicle 10 is 0°, the direction of the face of the driver 30 when facing upward is represented as an angle between 0° and 90°, while the direction of the face of the driver 30 when facing downward is represented as an angle between 0° and −90°.

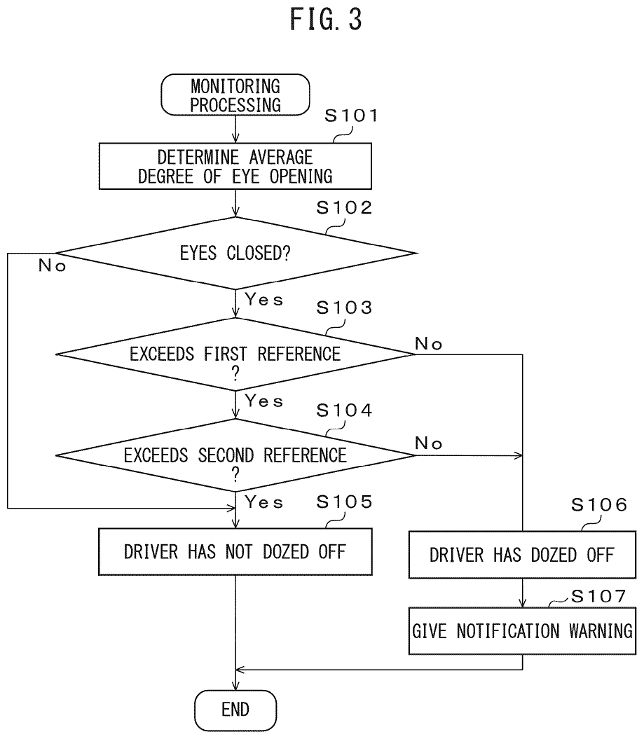

FIG. 3 is an example of an operation flow chart for monitoring processing by the monitoring device 11 of the embodiment. Monitoring processing by the monitoring device 11 will be described below with reference to FIG. 3. The monitoring device 11 carries out monitoring processing according to the operation flow chart shown in FIG. 3, at a monitoring time with a predetermined cycle.

First, the determining unit 232 calculates the average value for the degree of eye opening of the driver 30 (step S101). For example, the determining unit 232 calculates the average degree of eye opening obtained within the most recent fixed time period for both the left and right eyes of the driver 30. The most recent fixed time period may be 10 to 30 seconds.

The determining unit 232 then determines whether or not the eyes of the driver 30 are closed based on the degree of eye opening of the driver 30 (step S102). For example, when the average degrees of eye opening for the left and right eyes of the driver 30 are both at or below the reference degree of eye opening, the determining unit 232 determines that the eyes of the driver 30 are closed. The reference degree of eye opening may be 3 mm, for example. The degree of eye opening of a human is usually in the range of 10 mm to 30 mm.

On the other hand, when the average degree of eye opening for either one of the left and right eyes of the driver 30 is not at or below the reference degree of eye opening, the determining unit 232 determines that the eyes of the driver 30 are not closed.

When it has been determined that the eyes of the driver 30 are closed (step S102—Yes), the determining unit 232 determines whether or not the direction of the face of the driver 30 indicates movement in the right direction exceeding the first reference, based on information representing movement of the face of the driver 30 within a predetermined determining period (step S103). The determining period may be 1 to 5 seconds, for example. The determining period may start from the time at which it has been determined that the eyes of the driver 30 are closed. The determining period may also include the time at which it has been determined that the eyes of the driver 30 are closed.

When the information representing movement of the face of the driver 30 is represented as an angle, the first reference may be −10°, for example. When the information representing movement of the face of the driver 30 is represented as angular velocity, the first reference may be −2.5° /sec, for example. When the information representing movement of the face of the driver 30 is represented as angular acceleration, the first reference may be −2.5° /sec², for example.

The determining unit 232 examines the information representing movement of the face of the driver 30 from the start time of the determining period. When a value exceeds the first reference for information representing movement of the face of the driver 30, the determining unit 232 determines that the direction of the face of the driver 30 indicates movement in the right direction exceeding the first reference.

When the direction of the face of the driver 30 indicates movement in the right direction exceeding the first reference (hereunder also referred to as “first action”) (step S103—Yes), the determining unit 232 determines whether or not the direction of the face of the driver 30 indicates movement in the left direction exceeding the second reference, in continuation with the first action, based on the information representing movement of the face of the driver 30 for the determining period (step S104). The determining period is the same period as for determination of the first action. The information representing movement of the face of the driver 30 is the same information as for the first action.

The determining unit 232 examines the information representing movement of the face of the driver 30 from the start time of the determining period. When a value exceeds the second reference for information representing movement of the face of the driver 30, in continuation with the first action, the determining unit 232 determines that the direction of the face of the driver 30 indicates movement in the left direction exceeding the second reference (hereunder also referred to as “second action”). The second action may sometimes occur before the first action, or it may sometimes occur after the first action.

When the information representing movement of the face of the driver 30 is represented as an angle, the second reference may be 10°, for example. When the information representing movement of the face of the driver 30 is represented as angular velocity, the second reference may be +2.5° /sec, for example. When the information representing movement of the face of the driver 30 is represented as angular acceleration, the second reference may be +2.5°/sec², for example. The absolute value of the second reference may be either the same as or different from the first reference.

When the direction of the face of the driver 30 indicates movement in the left direction exceeding the second reference (step S104—Yes) or it has been determined that the eyes of the driver 30 are not closed (step S102—No), the determining unit 232 determines that the driver 30 has not dozed off (step S105), and the series of processing steps is complete.

On the other hand, when the direction of the face of the driver 30 does not indicate movement in the right direction exceeding the first reference (step S103—No), or the direction of the face of the driver 30 does not indicate movement in the left direction exceeding the second reference (step S104—No), the determining unit 232 determines that the driver 30 has dozed off (step S106).

The processing to determine whether or not the eyes are closed may also be carried out between step S103 and step S104, and step S106. After the determining unit 232 has determined that the eyes of the driver 30 are closed during a predetermined time period (such as 3 to 10 seconds), it may proceed to step S106. When it has been determined during this period that the eyes of the driver 30 are not closed, the series of processing steps is complete.

A situation in which it has not been determined that the direction of the face of the driver 30 indicates movement in the right direction exceeding the first reference and, in continuation with the movement in the right direction, the direction of the face of the driver 30 indicates movement in the left direction exceeding the second reference, includes situations in which there is no first action or second action, there is a first action but no second action, and there is no first action but there is a second action.

The control unit 233 then gives the driver 30 a notification warning via the UI 4 (step S107), and the series of processing steps is complete. For example, the control unit 233 outputs a loud alarm while displaying a warning on the display device 4a, via the UI 4. The monitoring device 11 thus increases the degree of participation of the driver 30 in driving.

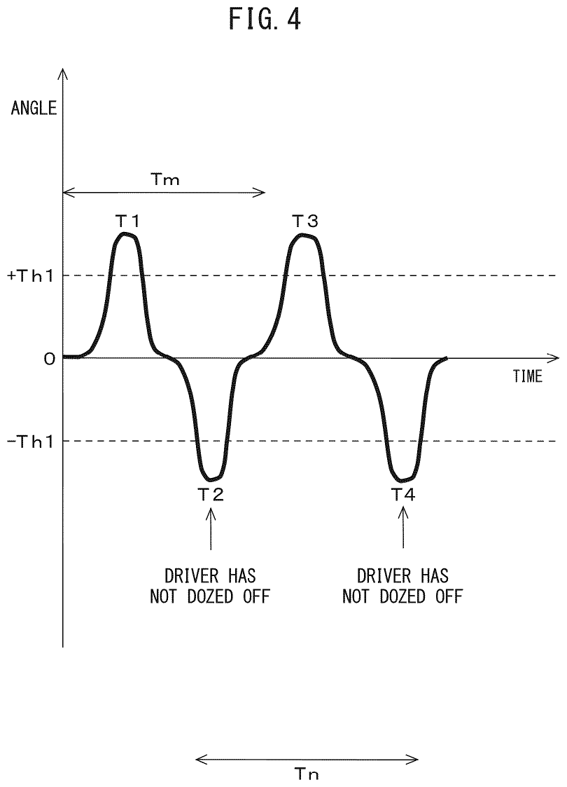

FIG. 4 is a diagram showing change in facial direction representing movement of the face of the driver 30. The ordinate represents the angle of the facial direction in the horizontal direction, and the abscissa represents time. It is determined that the eyes of the driver 30 are closed at time zero. The determining unit 232 determines the movement of the face of the driver 30 from time zero throughout a time period Tm.

After the angle representing the facial direction has exceeded (first reference+Th1) at time T1, it exceeds (second reference−Th1) at time T2. The second action wherein the angle exceeds (second reference−Th1) at time T2 is in continuation with the first action where the angle exceeds (first reference+Th1) at time T1. Time T2 is within the time period Tm. The driver 30 is estimated to be smiling. The determining unit 232 therefore determines that the driver 30 has not dozed off.

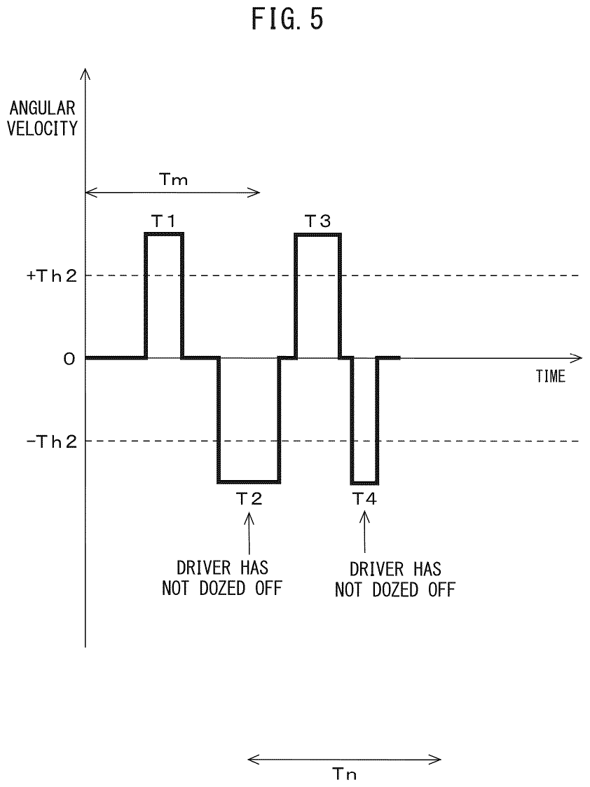

FIG. 5 is a diagram showing change in angular velocity representing movement of the face of the driver 30. The ordinate represents the angular velocity of the facial direction in the horizontal direction, and the abscissa represents time. It is determined that the eyes of the driver 30 are closed at time zero. The determining unit 232 determines movement of the face of the driver 30 from time zero throughout time period Tm.

After the angular velocity representing facial movement has exceeded (first reference+Th2) at time T1, it exceeds (second reference−Th2) at time T2. The second action wherein the angular velocity exceeds (second reference−Th2) at time T2 is in continuation with the first action where the angular velocity exceeds (first reference+Th2) at time T1. Time T2 is within the time period Tm. The driver 30 is estimated to be smiling. The determining unit 232 therefore determines that the driver 30 has not dozed off.

To represent the angle of the facial direction it is necessary to set the traveling direction of the vehicle 10 as a reference angle. The angular velocity, on the other hand, being represented by change in the angle per unit time, requires no reference, and therefore processing using the angular velocity is simpler. Processing is likewise simpler when the movement of the face of the driver 30 is represented as angular acceleration.

The monitoring device of the embodiment described in detail above can determine that the driver has dozed off without erroneously detecting a smiling face.

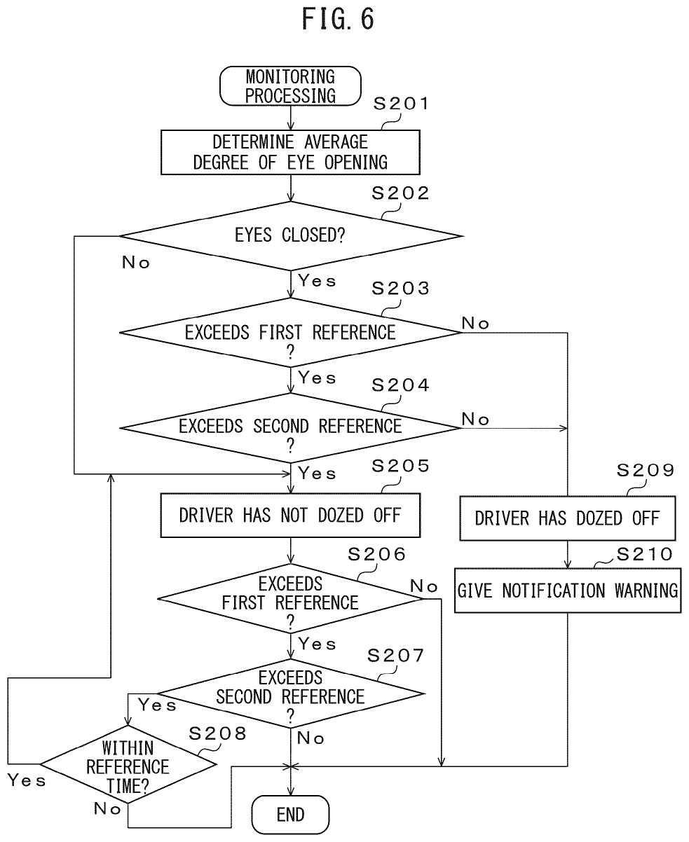

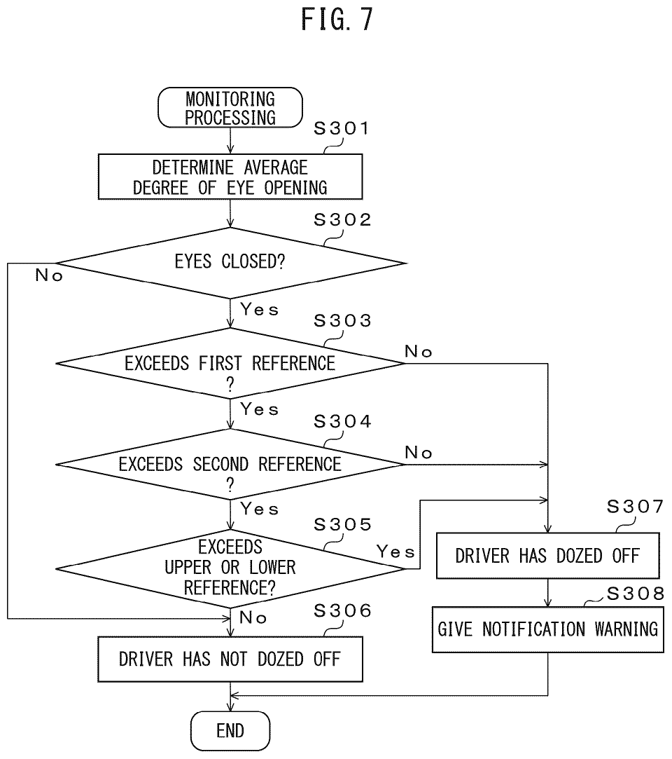

Monitoring devices of the second and third embodiments disclosed herein will now be explained with reference to FIG. 6 and FIG. 7. The explanation for the monitoring device of the first embodiment applies for any aspects of the monitoring devices of these embodiments that are not explained here.

FIG. 6 is an example of an operation flow chart for monitoring processing by the monitoring device 11 of the second embodiment. This embodiment differs from the first embodiment described above in that the processing of steps S206 to S208 are added. The processing for steps S201 to S205, S209 and S210 are the same as in steps S101 to S107 described above.

For this embodiment, after it has been determined that the driver 30 has not dozed off (step S205), the determining unit 232 determines whether or not the direction of the face of the driver 30 indicates movement in the right direction exceeding the first reference (step S206).

The determining unit 232 determines whether or not the direction of the face of the driver 30 indicates movement in the right direction exceeding the first reference, based on information representing movement of the face of the driver 30 within a predetermined determining period. The determining period may be 1 to 5 seconds, for example. The determining period may start from the time at which it has been determined that the driver 30 has not dozed off.

When the direction of the face of the driver 30 indicates movement in the right direction exceeding the first reference (step S206—Yes), the determining unit 232 determines whether or not the direction of the face of the driver 30 indicates movement in the left direction exceeding the second reference, in continuation with the first action, based on the information representing movement of the face of the driver 30 for the determining period (step S207).

When the direction of the face of the driver 30 indicates movement in the left direction exceeding the second reference (step S207—Yes), the determining unit 232 determines whether or not it is within the reference time from the time at which it was determined that the driver 30 has not dozed off (step S208). The determining unit 232 determines whether or not the time at which the second action was confirmed is within the reference time from the time at which it was determined that the driver 30 has not dozed off. The reference time may be 3 to 10 seconds, for example.

When the time is within the reference time (step S208—Yes), the determining unit 232 determines that the driver 30 has not dozed off (step S205). Processing then proceeds to step S206.

On the other hand, when the direction of the face of the driver 30 does not indicate movement in the right direction exceeding the first reference (step S206—No), or the direction of the face of the driver 30 does not indicate movement in the left direction exceeding the second reference (step S207—No), or the time is not within the reference time (step S208—No), the series of processing steps is complete. Since it is estimated that the driver 30 is not smiling, the monitoring processing returns again to begin from monitoring a closed-eye state.

Monitoring processing of the embodiment will now be explained with reference to FIG. 4 and FIG. 5. In the example shown in FIG. 4, first it is determined at time T2 that the driver 30 has not dozed off. The determining unit 232 therefore continues to determine movement of the face of the driver 30, without determining a closed-eye state.

After the angle representing the facial direction has exceeded (first reference+Th1) at time T3, it exceeds (second reference−Th1) at time T4. The second action wherein the angle exceeds (second reference−Th1) at time T4 is in continuation with the first action where the angle exceeds (first reference+Th1) at time T3.

Time T4 is within the reference time Tn from time T2 at which it was determined that the driver 30 has not dozed off. The determining unit 232 therefore determines that the driver 30 has not dozed off.

In the example shown in FIG. 5, first it is determined at time T2 that the driver 30 has not dozed off. The determining unit 232 therefore continues to determine movement of the face of the driver 30, without determining a closed-eye state.

After the angular velocity representing facial movement has exceeded (first reference+Th2) at time T3, it exceeds (second reference−Th2) at time T4. The second action where the angular velocity exceeds (second reference−Th2) at time T4 is in continuation with the first action where the angular velocity exceeds (first reference+Th2) at time T3.

Time T4 is within the reference time Tn from time T2 at which it was determined that the driver 30 has not dozed off. The determining unit 232 therefore determines that the driver 30 has not dozed off.

For this embodiment, when it has been determined that the driver 30 has not dozed off, the driver 30 is estimated to be smiling. For this embodiment, therefore, movement of the face of the driver 30 continues to be determined without determining a closed-eye state.

As explained in detail above, the monitoring device of this embodiment can reduce processing volume by the monitoring device. Moreover the monitoring device of this embodiment exhibits the same effect as the first embodiment.

FIG. 7 is an example of an operation flow chart for monitoring processing by the monitoring device 11 of the third embodiment. This embodiment differs from the first embodiment described above in that the processing of step S305 is added. The processing for steps S301 to S304, S306 and S308 are the same as in steps S101 to S107 described above.

When the direction of the face of the driver 30 indicates movement in the left direction exceeding the second reference (step S304—Yes), the determining unit 232 determines whether or not the direction of the face of the driver 30 indicates movement in the upward direction exceeding the third reference, or the direction of the face of the driver 30 indicates movement in the downward direction exceeding the fourth reference, based on movement of the face of the driver 30 (step S305).

The determining unit 232 acquires information representing movement of the face of the driver 30 in the vertical direction from the detecting unit 231. The information representing movement of the face of the driver 30 in the vertical direction is represented as an angle, angular velocity or angular acceleration.

The determining unit 232 determines whether or not the direction of the face of the driver 30 indicates movement in the upward direction exceeding the third reference, or the direction of the face of the driver 30 indicates movement in the downward direction exceeding the fourth reference, based on information representing movement of the face of the driver 30 in the vertical direction. The explanation for processing to determine information representing movement of the face of the driver 30 in the horizontal direction is applied as appropriate for determining information representing movement of the face of the driver 30 in the vertical direction. For example, the third reference may be 10°, and the fourth reference may be −10°.

When the direction of the face of the driver 30 indicates movement in the upward direction exceeding the third reference, or the direction of the face of the driver 30 indicates movement in the downward direction exceeding the fourth reference (step S305—Yes), the determining unit 232 determines that the driver 30 has dozed off (step S307).

Since it is highly likely that the driver 30 has dozed off if the direction of the face of the driver 30 has indicated movement in the upward direction or downward direction exceeding the reference, it is determined that the driver 30 has dozed off regardless of movement of the face of the driver 30 in the horizontal direction.

As explained in detail above, the monitoring device of this embodiment can reliably determine if the driver 30 has dozed off. Moreover the monitoring device of this embodiment exhibits the same effect as the first embodiment.

The determining device, computer program for determination and determining method according to the embodiments described in the present disclosure may incorporate appropriate modifications that still fall within the gist of the disclosure. Moreover, the technical scope of the disclosure is not limited to these embodiments, and includes the present disclosure and its equivalents as laid out in the Claims.

For example, processing for calculation of the degree of eye opening of the driver as described in the embodiments is merely an example, and is not intended to be limitative. Processing for determining movement of the driver's face as described in the embodiments is also merely an example and is not intended to be limitative.

1. A determining device comprising:
a processor configured to
determine whether a degree of eye opening of a driver is at or below a reference degree of eye opening,
determine whether a direction of a driver's face indicates movement in a right direction exceeding a first reference and, in continuation with the movement in the right direction, the direction of the driver's face indicates movement in a left direction exceeding a second reference, based on information representing movement of the driver's face, and
determine that the driver has not dozed off when it has been determined that the degree of eye opening of the driver is at or below the reference degree of eye opening and it has been determined that the direction of the driver's face indicates movement in the right direction exceeding the first reference and, in continuation with the movement in the right direction, the direction of the driver's face indicates movement in the left direction exceeding the second reference, and determine that the driver has dozed off when it has been determined that the degree of eye opening of the driver is at or below the reference degree of eye opening and it has not been determined that the direction of the driver's face indicates movement in the right direction exceeding the first reference and, in continuation with the movement in the right direction, the direction of the driver's face indicates movement in the left direction exceeding the second reference.

2. The determining device according to claim 1, wherein the information representing movement of the driver's face is represented by an angle of the direction of the driver's face in a horizontal direction, angular velocity of the direction of the driver's face in the horizontal direction, or angular acceleration of the direction of the driver's face in the horizontal direction.

3. The determining device according to claim 1, wherein the processor is further configured to determine that the driver has not dozed off when it has been determined that the direction of the driver's face indicates movement in the right direction exceeding the first reference and, in continuation with the movement in the right direction, the direction of the driver's face indicates movement in the left direction exceeding the second reference, within a reference time from the time at which it has determined that the driver has not dozed off.

4. The determining device according to claim 1, wherein the processor is further configured to
determine whether the direction of the driver's face indicates movement in an upward direction exceeding a third reference or the direction of the driver's face indicates movement in a downward direction exceeding a fourth reference, based on movement of the driver's face, and
determine that the driver has dozed off when it has been determined that the degree of eye opening of the driver is at or below the reference degree of eye opening and it has been determined that the direction of the driver's face indicates movement in the upward direction exceeding the third reference or the direction of the driver's face indicates movement in the downward direction exceeding the fourth reference, even when it has been determined that the direction of the driver's face indicates movement in the right direction exceeding the first reference and, in continuation with the movement in the right direction, the direction of the driver's face indicates movement in the left direction exceeding the second reference.

5. A computer-readable, non-transitory storage medium storing a computer program for determination, which causes a processor to execute a process, and the process comprises:
determining whether a degree of eye opening of a driver is at or below a reference degree of eye opening;
determining whether a direction of a driver's face indicates movement in a right direction exceeding a first reference and, in continuation with the movement in the right direction, the direction of the driver's face indicates movement in a left direction exceeding a second reference, based on information representing movement of the driver's face; and
determining that the driver has not dozed off when it has been determined that the degree of eye opening of the driver is at or below the reference degree of eye opening and it has been determined that the direction of the driver's face indicates movement in the right direction exceeding the first reference and, in continuation with the movement in the right direction, the direction of the driver's face indicates movement in the left direction exceeding the second reference, and determining that the driver has dozed off when it has been determined that the degree of eye opening of the driver is at or below the reference degree of eye opening and it has not been determined that the direction of the driver's face indicates movement in the right direction exceeding the first reference and, in continuation with the movement in the right direction, the direction of the driver's face indicates movement in the left direction exceeding the second reference.

6. A determining method carried out by a determining device and the method comprises:
determining whether a degree of eye opening of a driver is at or below a reference degree of eye opening,
determining whether a degree of eye opening of a driver is at or below a reference degree of eye opening;
determining whether a direction of a driver's face indicates movement in a right direction exceeding a first reference and, in continuation with the movement in the right direction, the direction of the driver's face indicates movement in a left direction exceeding a second reference, based on information representing movement of the driver's face; and
determining that the driver has not dozed off when it has been determined that the degree of eye opening of the driver is at or below the reference degree of eye opening and it has been determined that the direction of the driver's face indicates movement in the right direction exceeding the first reference and, in continuation with the movement in the right direction, the direction of the driver's face indicates movement in the left direction exceeding the second reference, and determining that the driver has dozed off when it has been determined that the degree of eye opening of the driver is at or below the reference degree of eye opening and it has not been determined that the direction of the driver's face indicates movement in the right direction exceeding the first reference and, in continuation with the movement in the right direction, the direction of the driver's face indicates movement in the left direction exceeding the second reference.
